(19) United States
(12) Patent Application Publication
(10) Pub. No.: US 2026/0232393 A1
(43) Pub. Date: Aug. 13, 2026

(54) SURGICAL AUXILIARY DEVICE

(71) Applicant: Point Robotics Medtech Inc., Hsinchu County (TW)

(72) Inventor: Ren-Jeng Wang, Hsinchu County (TW)

(21) Appl. No.: 19/277,356

(22) Filed: Jul. 22, 2025

(30) Foreign Application Priority Data
Feb. 11, 2025 (TW) 114104928

(51) Int. Cl. A61B 34/30

(52) U.S. Cl. CPC A61B 34/30; A61B 2034/304

(57) ABSTRACT

A surgical auxiliary device includes a base body, an active robotic arm, and two passive robotic arms. The active robotic arm is disposed on the base body. The two passive robotic arms are disposed on two sides of the base body, respectively. A highest position of each of the passive robotic arms in a passive initial posture is lower than a lowest position of the active robotic arm in an active initial posture.

### CROSS-REFERENCE TO RELATED PATENT APPLICATION

This application claims the benefit of priority to Taiwan Patent Application No. 114104928, filed on Feb. 11, 2025. The entire content of the above identified application is incorporated herein by reference.

### FIELD OF THE DISCLOSURE

The present disclosure relates to an auxiliary device, and more particularly to a surgical auxiliary device.

### BACKGROUND OF THE DISCLOSURE

Currently, a surgeon uses a surgical robot to assist in performing surgical procedures. Generally, a surgical robot has more than one robotic arms mounted on a base platform of the surgical robot, such that the robotic arms can cooperate with the surgeon.

However, since the inner space of the operating room is quite small, a working area for the surgeon to perform operations may be squeezed by the surgical robot occupying a portion of the space. Additionally, each robotic arm of the surgical robot includes multiple moving and rotating mechanism components which facilitate multi-degree-of-freedom motion. Consequently, simultaneous actuation of the multiple robotic arms may easily incur a high risk of mutual interference and collision.

Therefore, how to overcome the above-mentioned problems through improvements in structural design has become an important issue to be addressed in the related art.

### SUMMARY OF THE DISCLOSURE

In response to the above-referenced technical inadequacies, the present disclosure provides a surgical auxiliary device that is capable of reducing a storage space of robotic arms and preventing the robotic arms from interfering with each other.

In order to solve the above-mentioned problems, one of the technical aspects adopted by the present disclosure is to provide a surgical auxiliary device, which includes a base body, an active robotic arm, and two passive robotic arms. The active robotic arm is disposed on the base body. The two passive robotic arms are disposed on two sides of the base body, respectively. A highest position of each of the passive robotic arms in a passive initial posture is lower than a lowest position of the active robotic arm in an active initial posture.

Therefore, in the surgical auxiliary device provided by the present disclosure, the highest position of each of the passive robotic arms in the passive initial posture is lower than the lowest position of the active robotic arm in the active initial posture, the active robotic arm and the passive robotic arms do not interfere and collide with each other during simultaneous actuation.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a surgical auxiliary device according to an embodiment of the present disclosure;

FIG. 2 is a schematic side view of the surgical auxiliary device according to the embodiment of the present disclosure;

FIG. 3 is another schematic perspective view of the surgical auxiliary device according to the embodiment of the present disclosure;

FIG. 4 is a functional block diagram of the surgical auxiliary device according to the embodiment of the present disclosure;

FIG. 5 is a schematic perspective view of an active robotic arm of the surgical auxiliary device according to the embodiment of the present disclosure; and

FIG. 6 is a schematic perspective view of a passive robotic arm of the surgical auxiliary device according to the embodiment of the present disclosure.

### DETAILED DESCRIPTION OF THE EXEMPLARY EMBODIMENTS

### Embodiment

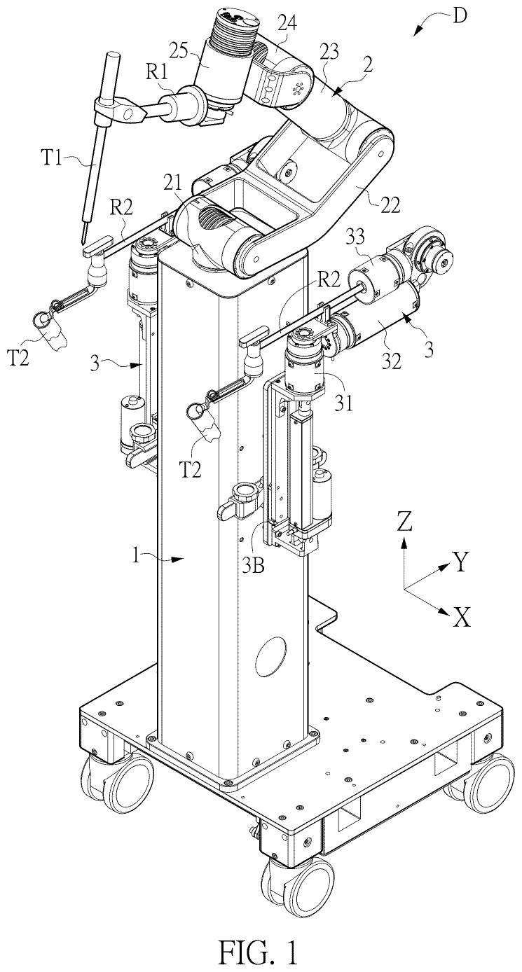

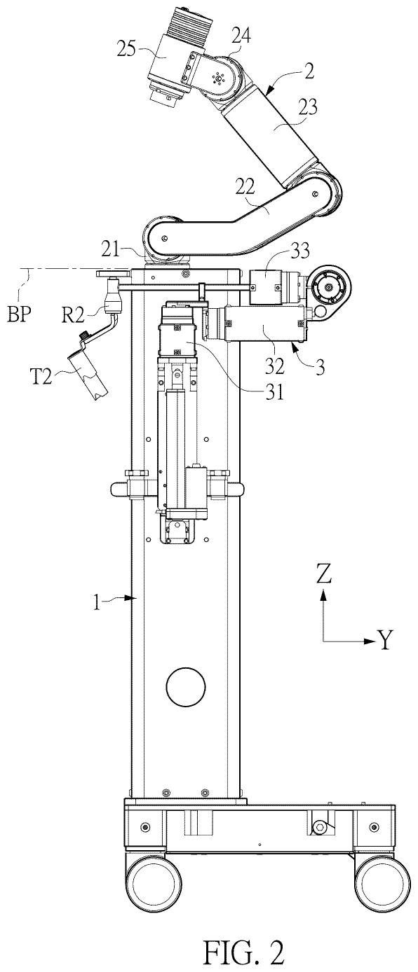

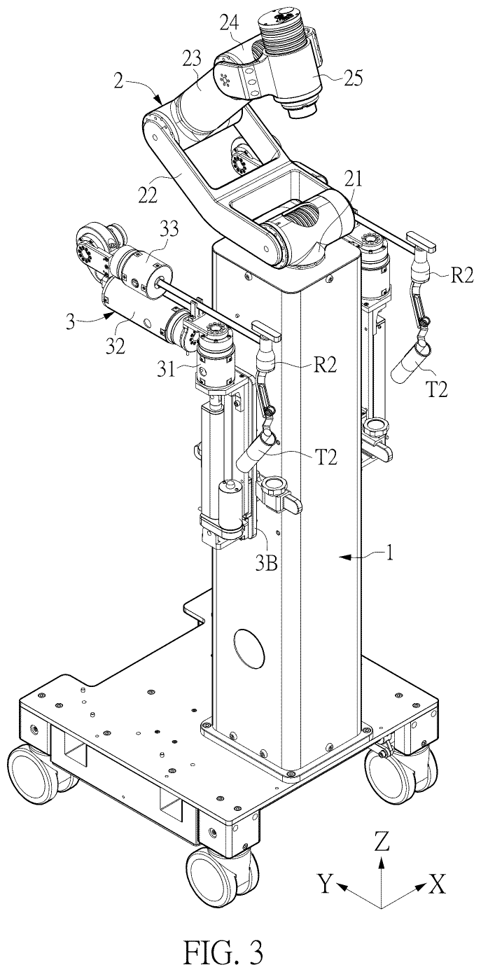

Reference is made to FIG. 1 to FIG. 3. FIG. 1 and FIG. 3 are different schematic perspective views of a surgical auxiliary device according to an embodiment of the present disclosure. FIG. 2 is a schematic side view of the surgical auxiliary device according to the embodiment of the present disclosure. The present disclosure provides a surgical auxiliary device D, which includes a base body 1, an active robotic arm 2, and two passive robotic arms 3. The active robotic arm 2 and the two passive robotic arms 3 are disposed on the base body 1. The two passive robotic arms are disposed on two sides of the base body 1, respectively. Quantities of the active robotic arm 2 and the passive robotic arms 3 are not limited in the present disclosure. In addition, it should be noted that a clamping arm R1, a holder R2, and surgical tools T1 and T2 are omitted in FIGS. 2 and 3.

In the present disclosure, the active robotic arm 2 can automatically move and/or rotate upon receiving commands that are input by a user (e.g., a surgeon), whereas the passive robotic arms 3 require be moved and/or rotated under manual operation of the user (i.e., the user manually moves the passive robotic arms 3).

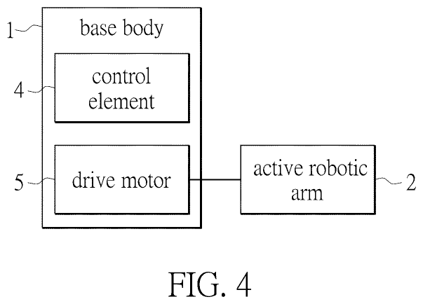

Reference is made to FIG. 3 and FIG. 4. FIG. 4 is a functional block diagram of the surgical auxiliary device according to the embodiment of the present disclosure. The surgical auxiliary device D further includes a control element 4 and a drive motor 5. The control element 4 and the drive motor 5 are disposed inside the base body 1, the control element 4 is electrically connected to the drive motor 5, and the drive motor 5 is connected to the active robotic arm 2. The control element 4 may include, but is not limited to, a processor, memory, and an input/output interface (not shown in the figures). The processor can be a central processor or an integrated circuit, such as a programmable logic controller (PLC) circuit, a microprocessor circuit, or a microcontroller circuit. The memory may be any storage device used to store data, such as random-access memory (RAM), read-only memory (ROM), flash memory, or a hard drive. The input/output interface may include physical connection ports and wireless communication modules (e.g., interface cards supporting Bluetooth® or Wi-Fi®). The control element 4 can be electrically connected to an external control interface through the input/output interface. Through the control interface, the user can input commands into the control element 4 to control the drive motor 5, thereby driving the active robotic arm 2 to automatically move or rotate.

As shown in FIGS. 1 to 3, the position of each of the passive robotic arms 3 is lower than that of the active robotic arm 2. Furthermore, the active robotic arm 2 and the passive robotic arms 3 shown in FIGS. 1 to 3 are all in their initial postures (i.e., the postures before actuation). For convenience of illustration, the initial posture of the active robotic arm 2 can be referred to as an active initial posture, and the initial posture of the passive robotic arms 3 can be referred to as a passive initial posture. A highest position of each of the passive robotic arms 3 in the passive initial posture is lower than a lowest position of the active robotic arm 2 in the active initial posture.

As shown in FIG. 2, a top portion of the base body 1 defines a reference plane BP. The lowest position of the active robotic arm 2 in the active initial posture is above the reference plane BP, and the highest position of each of the passive robotic arms 3 in the passive initial posture is below the reference plane BP. The space above the reference plane BP can be considered as a predetermined movement range of the active robotic arm 2, and the space below the reference plane BP can be considered as a predetermined movement range of the passive robotic arms 3. Therefore, by mechanically configuring the lowest position of the active robotic arm 2 in the active initial posture to be above the reference plane BP, and by mechanically configuring the highest position of each of the passive robotic arms 3 in the passive initial posture to be below the reference plane BP, the predetermined movement range of the active robotic arm 2 and that of the passive robotic arms 3 do not overlap with each other, thereby reducing the probability of interference and collision between the active robotic arm 2 and the passive robotic arms 3.

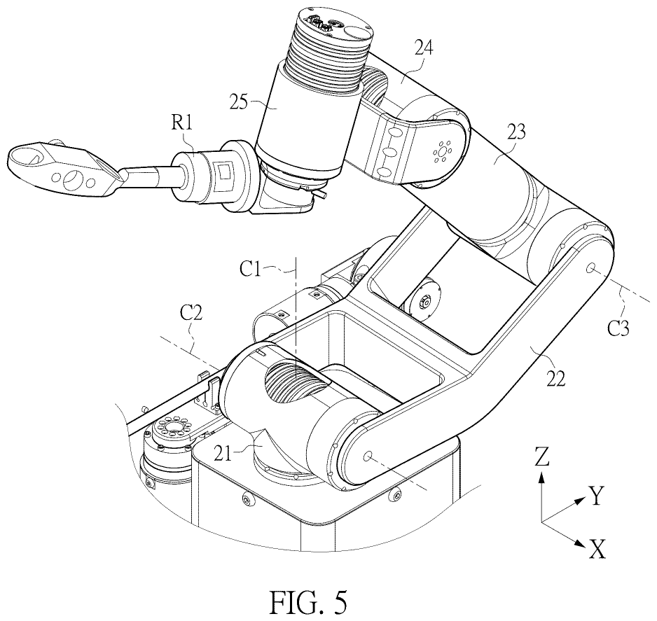

In the present disclosure, each of the active robotic arm 2 and the passive robotic arms 3 consists of multiple joint components and multiple link components, and thereby can perform actions such as rotation, extension, and retraction. Reference is made to FIGS. 1, 2, and 5. FIG. 5 is a schematic perspective view of an active robotic arm of the surgical auxiliary device according to the embodiment of the present disclosure. For example, the active robotic arm 2 includes a first limb 21, a second limb 22, and a third limb 23. The first limb 21 is connected to the top portion of the base body 1, and the second limb 22 is connected between the first limb 21 and the third limb 23. The first limb 21 spins about a first rotation axis C1, which is parallel to a Z-axis. That is, the first limb 21 is coaxial with the first rotation axis C1. The first rotation axis C1 is perpendicular to the reference plane BP. The second limb 22 rotates about a second rotation axis C2, and the second rotation axis C2 is parallel to an X-axis as the active robotic arm 2 is in the active initial posture. That is, the second limb 22 is perpendicular to the second rotation axis C2. The third limb 23 rotates about a third rotation axis C3, and the third rotation axis C3 is parallel to the X-axis as the active robotic arm 2 is in the active initial posture. That is, the third limb 23 is perpendicular to the third rotation axis C3.

Additionally, the active robotic arm 2 further includes a fourth limb 24 and a fifth limb 25. The fourth limb 24 is connected to the fifth limb 25, and is disposed between the third limb 23 and the fifth limb 25. The fourth limb 24 is parallel to the third rotation axis C3, and the fifth limb 25 is perpendicular to the third rotation axis C3. The fifth limb 25 can be optionally connected to other components. For example, the clamping arm R1 can be detachably connected to the fifth limb 25, and the clamping arm R1 can hold the surgical tool T1 (e.g., a surgical drill).

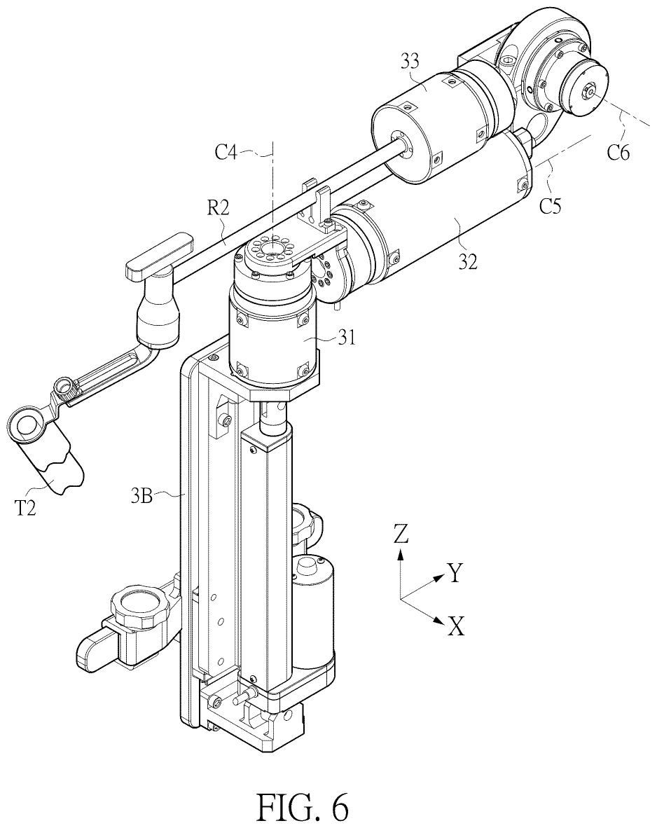

Reference is made to FIGS. 1, 2, and 6. FIG. 6 is a schematic perspective view of a passive robotic arm of the surgical auxiliary device according to the embodiment of the present disclosure. For example, each of the passive robotic arms 3 includes a mount 3B, a first limb 31, a second limb 32, and a third limb 33. The mount 3B is connected to a side surface of the base body 1, the first limb 31 is connected to a top portion of the mount 3B, and the second limb 32 is connected between the first limb 31 and the third limb 33. The first limb 31 spins about a fourth rotation axis C4, and the fourth rotation axis C4 is parallel to the Z-axis. That is, the first limb 31 is coaxial with the fourth rotation axis C4. The fourth rotation axis C4 is perpendicular to the reference plane BP. The second limb 32 spins about a fifth rotation axis C5, and the fifth rotation axis C5 is parallel to a Y-axis as the passive robotic arm 3 in the passive initial posture. That is, the second limb 32 is coaxial with the fifth rotation axis C5. The second limb 32 and the fifth rotation axis C5 are both perpendicular to the fourth rotation axis C4. The fourth rotation axis C4 is perpendicular to the fifth rotation axis C5. The third limb 33 rotates about a sixth rotation axis C6, and the sixth rotation axis C6 is parallel to the X-axis as the passive robotic arm 3 in the passive initial posture. The third limb 33 is perpendicular to the sixth rotation axis C6. In other words, as shown in FIG. 6, when each of the passive robotic arms 3 is in the passive initial posture, the second limb 32 is folded and disposed beneath the third limb 33, such that the second limb 32 is parallel to the third limb 33. Additionally, the third limb 33 of each of the passive robotic arms 3 can be connected to the holder R2 which can be used to secure another surgical tool T2 (e.g., an endoscope assembly).

By mechanically configuring the second limb 22 of the active robotic arm 2 to be perpendicular to the second rotation axis C2, the active robotic arm 2 actuates relative to the base body 1, so that the active robotic arm 2 can automatically extend upward or retract downward in a direction perpendicular to the second rotation axis C2 (i.e., along the Z-axis) or in a direction along the Y-axis. Furthermore, by mechanically configuring the second limb 32 of each of the passive robotic arms 3 to be coaxial with the fifth rotation axis C5, and by mechanically configuring both of the second limb 32 and the fifth rotation axis C5 to be perpendicular to the fourth rotation axis C4, the user can manually rotate the second limb 32 of each of the passive robotic arms 3 relative to the base body 1 in an outward manner (i.e., away from the base body 1), and then manually extend the passive robotic arms 3. After the surgery is completed, the user can manually rotate the second limb 32 of each of the passive robotic arms 3 in an inward manner (i.e., toward the base body 1) to fold the passive robotic arms 3.

Although the space above the reference plane BP can be considered as the predetermined movement range of the active robotic arm 2, and the space below the reference plane BP can be considered as the predetermined movement range of the passive robotic arms 3, the active robotic arm 2 may still extend into the space below the reference plane BP, and the passive robotic arms 3 may still extend into the space above the reference plane BP. However, since the actuation of the active robotic arm 2 is automatic, and the passive robotic arms 3 are manually controlled by the user, the user can coordinate the automatic actuation of the active robotic arm 2 with the manual operation of the passive robotic arms 3, thereby preventing the passive robotic arms 3 from reaching to an extension range of the active robotic arm 2. Accordingly, interference between the active robotic arm 2 and the passive robotic arms 3 can be avoided.

### Beneficial Effects of the Embodiment

In conclusion, in the surgical auxiliary device D provided by the present disclosure, by mechanically configuring the lowest position of the active robotic arm 2 in the active initial posture to be above the reference plane BP, and by mechanically configuring the highest position of each of the passive robotic arms 3 in the passive initial posture to be below the reference plane BP, the predetermined movement range of the active robotic arm 2 and that of the passive robotic arms 3 do not overlap with each other, thereby reducing the probability of interference and collision between the active robotic arm 2 and the passive robotic arms 3. Moreover, since the active robotic arm 2 and the passive robotic arms 3 are respectively positioned above and below the reference plane BP, the volume of the surgical auxiliary device D during storage can also be minimized, so as to reduce the space occupied by the surgical auxiliary device D.

Furthermore, while the actuation of the active robotic arm 2 of the surgical auxiliary device D is automatic, the passive robotic arms 3 of the surgical auxiliary device D are manually controlled by the user. Hence, by mechanically configuring the second limb 22 of the active robotic arm 2 to be perpendicular to the second rotation axis C2, by mechanically configuring the second limb 32 of the passive robotic arms 3 to be coaxial with the fifth rotation axis C5, and by mechanically configuring both of the second limb 32 and the fifth rotation axis C5 to be perpendicular to the fourth rotation axis C4, the active robotic arm 2 automatically extends upward or retracts downward in the direction perpendicular to the second rotation axis C2 during actuation. The user can cooperate with the movement of the active robot arm 2 and manually control the second limb 32 of each of the passive robotic arms 3 to rotate in an outward manner (i.e., away from the base body 1), thereby preventing the passive robotic arms 3 from reaching to the extension range of the active robotic arm 2 and avoiding interference between the active robotic arm 2 and the passive robotic arms 3.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A surgical auxiliary device, comprising:
a base body;
an active robotic arm disposed on the base body; and
two passive robotic arms disposed on two sides of the base body, respectively, wherein each of the passive robotic arms includes a first limb, a second limb, and a third limb, the first limb is connected to a side surface of the base body, and the second limb is connected between the first limb and the third limb;
wherein a highest position of each of the passive robotic arms in a passive initial posture is lower than a lowest position of the active robotic arm in an active initial posture;
wherein, when each of the passive robotic arms is in the passive initial posture, the second limb is folded and disposed beneath the third limb, such that the second limb is parallel to the third limb.

2. The surgical auxiliary device according to claim 1, wherein a top portion of the base body defines a reference plane, the lowest position of the active robotic arm in the active initial posture is above the reference plane, and the highest position of each of the passive robotic arms in the passive initial posture is below the reference plane.

3. The surgical auxiliary device according to claim 2, wherein the active robotic arm includes a first limb, a second limb, and a third limb, the first limb of the active robotic arm is connected to the top portion of the base body, and the second limb of the active robotic arm is connected between the first limb of the active robotic arm and the third limb of the active robotic arm.

4. The surgical auxiliary device according to claim 3, wherein the first limb of the active robotic arm spins about a first rotation axis, the first limb of the active robotic arm is coaxial with the first rotation axis, and the first rotation axis is perpendicular to the reference plane.

5. The surgical auxiliary device according to claim 4, wherein the second limb of the active robotic arm rotates about a second rotation axis, and the second limb of the active robotic arm is perpendicular to the second rotation axis.

6. The surgical auxiliary device according to claim 5, wherein the third limb of the active robotic arm rotates about a third rotation axis, and the third limb of the active robotic arm is perpendicular to the third rotation axis.

7. The surgical auxiliary device according to claim 2, wherein the first limb of each of the passive robotic arms spins about a fourth rotation axis, the first limb of each of the passive robotic arms is coaxial with the fourth rotation axis, and the fourth rotation axis is perpendicular to the reference plane.

8. The surgical auxiliary device according to claim 7, wherein the second limb of each of the passive robotic arms spins about a fifth rotation axis, the second limb of each of the passive robotic arms is coaxial with the fifth rotation axis, and the fourth rotation axis is perpendicular to the fifth rotation axis.

9. The surgical auxiliary device according to claim 8, wherein the third limb of each of the passive robotic arms rotates about a sixth rotation axis, and the third limb of each of the passive robotic arms is perpendicular to the sixth rotation axis.

10. The surgical auxiliary device according to claim 1, further comprising a control element and a drive motor, wherein the control element is electrically connected to the drive motor, the drive motor is connected to the active robotic arm, and the drive motor is configured to be controlled by the control element for moving the active robotic arm.

11. The surgical auxiliary device according to claim 1, wherein the two passive robotic arms are manually moved.
